(19) United States
(12) Patent Application Publication
(10) Pub. No.: US 2026/0076790 A1
(43) Pub. Date: Mar. 19, 2026

(54) ENHANCED ORAL CARE IMPLEMENT AND ASSOCIATED METHODS

(71) Applicant: Carol Taurosa, Allenwood, NJ (US)

(72) Inventor: Carol Taurosa, Allenwood, NJ (US)

(21) Appl. No.: 19/307,223

(22) Filed: Aug. 22, 2025

Related U.S. Application Data
(60) Provisional application No. 63/695,668, filed on Sep. 17, 2024.

(51) Int. Cl. A61C 17/22; A46B 13/00; A46B 13/04; A46B 15/00; A61B 1/06; A61B 1/24; A61B 17/24; A61C 17/28

(52) U.S. Cl. CPC A61C 17/222; A46B 13/001; A46B 13/04; A46B 15/0036; A46B 15/0081; A61B 1/0684; A61B 1/24; A61B 17/244; A61C 17/227; A61C 17/28

(57) ABSTRACT

Described herein are enhanced oral care implements comprising a cylindrical or substantially cylindrical head component comprising bristles extending outward around the circumference of the head component from a wall element. The enhanced oral care implements described herein further comprise a neck component comprising a motor element and a power source; and a handle component.

### FIELD OF THE INVENTION

The present disclosure relates to an oral care implement designed to improve the efficiency and effectiveness of tooth brushing.

### BACKGROUND

Maintaining good oral hygiene enhances tooth longevity. An important element of oral hygiene is brushing teeth regularly and using optimal brushing techniques that allow for cleaning the entire tooth, including difficult to reach spaces between teeth. Improved oral hygiene achieved through more frequent, consistent, and effective tooth brushing, contributes to improved oral health and decreased health care costs ultimately.

Bacteria is present on all surfaces inside the mouth. The cheeks, tongue, and roof of the mouth are often overlooked in oral care. Failing to brush the cheeks, tongue, and roof of the mouth, in addition to the teeth and gums, creates a safe haven in which bacteria are able to rapidly proliferate. The National Institutes of Health states that accumulation of bacteria between the cheeks can cause decay to the connective tissue that holds your teeth. Also, if bacteria living in one's mouth are regularly swallowed it can lead to weakening of the immune system. It can also lead to osteoporosis and associated health issues. Moreover, many of the deleterious health effects identifiable with old age have been linked to poor oral hygiene.

Conventional toothbrushes are difficult to position in ways that are conducive to brushing the interior surface of teeth and the recesses between teeth. Further, given the often-immobile nature of a toothbrush head, it is left to the user to calibrate the speed and force used to brush. The self-monitoring nature of this process lends itself to frequent deleterious brushing habits such as applying too much force to teeth when brushing, brushing teeth using a sub-optimal motion, and applying too rapid a brushing motion. All of these deleterious brushing habits can lead to insufficient teeth cleaning as well as premature dislodging of teeth due to weakening of the gums and damage to tooth enamel. Insufficient cleaning of teeth, and more generally insufficient oral cavity cleaning, can lead to tooth decay, cavities, and the need for artificial replacement teeth or full dentures.

An additional reason that existing toothbrushes are sub-optimal is due to the limited amount of water retained in the bristles of the toothbrush head. Insufficient water on the toothbrush head when brushing diminishes the extent to which dentifrice-water solutions may reach the space between teeth, thus leaving the recesses between teeth vulnerable to formation and proliferation of bacteria. Such intra-oral bacteria are prime contributors to formation of cavities, and further contribute to contamination of the tooth pulp thereby necessitating a root canal procedure or risking tooth loss entirely. Failure to promptly and adequately address such matters may result in the bacteria infection spreading such as to the jaw, and may lead to dental abscesses, a stroke, sepsis, or a heart attack.

Therefore, a need exists to improve upon existing oral care implements to create an enhanced oral care implement designed to facilitate optimal tooth brushing technique and foster, as early in life as possible, frequent and thorough tooth brushing by making the tooth brushing process more efficient and effective. As an individual's oral care habits improve this will contribute to improved oral health, diminished risk of significant oral adverse events, decreased healthcare costs, and enhanced self-confidence that comes with having healthy-looking teeth. What is needed is an oral care implement comprising a head with bristles that completely surround the entire circumference of the brush head. What is needed is an oral care implement that comprises a flexible and motorized head that can be controlled with regard to brushing motion and pace of brushing. What is needed is an oral care implement comprising a supplemental source of water to facilitate tooth brushing, particularly with regard to spaces between teeth and other intra-oral locations that are difficult or nearly impossible to reach with existing oral care implements.

### SUMMARY

Described herein are enhanced oral care implements comprising a cylindrical or substantially cylindrical head component. The head component may include bristles extending outward around the circumference of the head component from a wall element. The oral care implement may further comprise a neck component that may include a motor element and a power source. In an embodiment, the neck component may also include a reservoir component, a pump element that includes a tubular aspect, and a valve element. The oral care implement may further comprise a handle component. In an embodiment, the handle component may include a tongue scraper aspect. In an embodiment, the handle component may comprise one or more light emitting diodes.

Also described herein are methods for cleaning the oral cavity of a human being comprising providing an enhanced oral care implement as described herein and using the enhanced oral care implement to clean the human being's oral cavity. In an embodiment, the methods may further comprise a step of scraping an individual's tongue using the tongue scraper described herein. In another embodiment, the methods may further comprise a step of using the one or more light emitting diodes to visually inspect an individual's oral cavity.

### BRIEF DESCRIPTION OF THE DRAWINGS

FIG. 1 is a front view of an enhanced oral care implement, according to an embodiment of this disclosure.

FIG. 2 is a front view of an enhanced oral care implement, showing a sectional view of the neck component thereof, according to an embodiment of this disclosure.

FIG. 3 is a front view of an enhanced oral care implement, showing a sectional view of the neck component thereof, according to an embodiment of this disclosure.

FIG. 4 is a front view of an enhanced oral care implement, showing a sectional view of the neck component thereof, according to an embodiment of this disclosure.

### DETAILED DESCRIPTION

The following disclosure is provided to describe various embodiments of an enhanced oral care element. Skilled artisans will appreciate additional embodiments and uses of the present invention that extend beyond the examples of this disclosure. Terms included by any claim are to be interpreted as defined within this disclosure. Singular forms should be read to contemplate and disclose plural alternatives. Similarly, plural forms should be read to contemplate and disclose singular alternatives. Conjunctions should be read as inclusive except where stated otherwise.

Terms and expressions used throughout this disclosure are to be interpreted broadly. Terms are intended to be understood respective to the definitions provided by this specification. Technical dictionaries and common meanings understood within the applicable art are intended to supplement these definitions. In instances where no suitable definition can be determined from the specification or technical dictionaries, such terms should be understood according to their plain and common meaning. However, any definitions provided by the specification will govern above all other sources.

Various objects, features, aspects, and advantages described by this disclosure will become more apparent from the following detailed description, along with the accompanying drawings in which like numerals represent like components.

Expressions such as “at least one of A, B, and C” should be read to permit any of A, B, or C singularly or in combination with the remaining elements. Additionally, such groups may include multiple instances of one or more element in that group, which may be included with other elements of the group. All numbers, measurements, and values are given as approximations unless expressly stated otherwise. Approximations are intended to indicate a value within 5% of the pertinent target value.

For the purpose of clearly describing the components and features discussed throughout this disclosure, some frequently used terms will now be defined, without limitation. The term “power source,” as it used throughout this disclosure, shall mean a lithium-ion re-chargeable battery or a non-rechargeable lithium, alkaline, or NiMH battery; however, other suitable alternatives may also be included within this definition as long as use of such alternatives is consistent with operability of the enhanced oral care implements described herein, as such would be readily discernible by those skilled in the art. The term “dentifrice,” as it is used throughout this disclosure shall mean a powder, paste, or liquid for cleaning teeth. The term “dentifrice-water solution,” as used throughout this disclosure, shall mean any mixture comprising between 15% and 95% (by mass) dentifrice in water. As used herein, the term “substantially” shall mean almost exactly or perfectly; specifically, within 1% of the target condition by all measurements.

Various aspects of the present disclosure will now be described in detail, without limitation. In the following disclosure, an enhanced oral care implement will be discussed. Those of skill in the art will appreciate alternative labeling of the enhanced oral care implement described herein such as a mechanically and/or diminesionally enhanced oral care implement, a mechanically and/or diminesionally enhanced toothbrush, an oral care implement having enhanced functionality, a toothbrush having enhanced functionality, the invention, or other similar names. Use of such alternative terminology should not be viewed as limiting in any way.

Described herein is an oral care implement that may comprise a cylindrical or substantially cylindrical head component. The head component may comprise a wall element comprising bristles protruding outward from the wall element. These outward protruding bristles may be located around the entire circumference of the head component. Oral care implements as described herein may comprise a neck component attached to the head component at a first end and attached to a handle component at a second end. The neck component may further comprise a motor element and a portable and rechargeable power source.

The neck component may further comprise a reservoir element configured to hold a solution. The head component may further comprise a nozzle element and a valve element. The valve element may operatively connect the head component with the reservoir element. The neck component may further comprise a pump element. The pump element may operatively connect the reservoir element with the nozzle element through the valve element. Solution housed within the reservoir element may be transferred through the nozzle element by the pump element, which may propel the solution through the valve element and then through the nozzle element. The solution may then be ejected out of the nozzle element into the surrounding environment, which may be a human being's oral cavity.

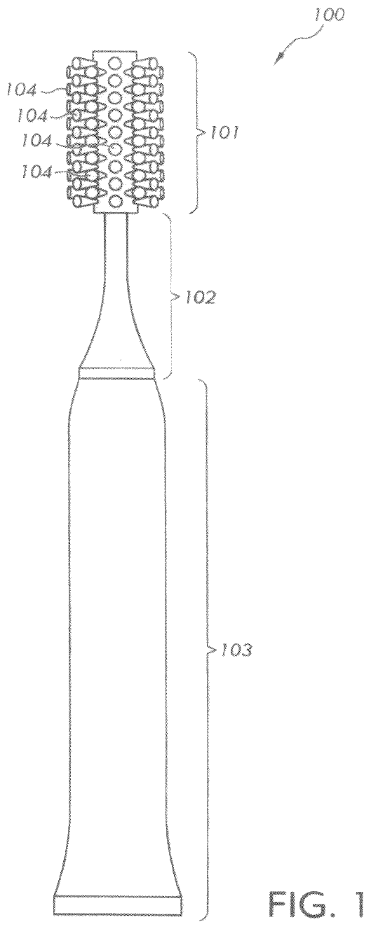

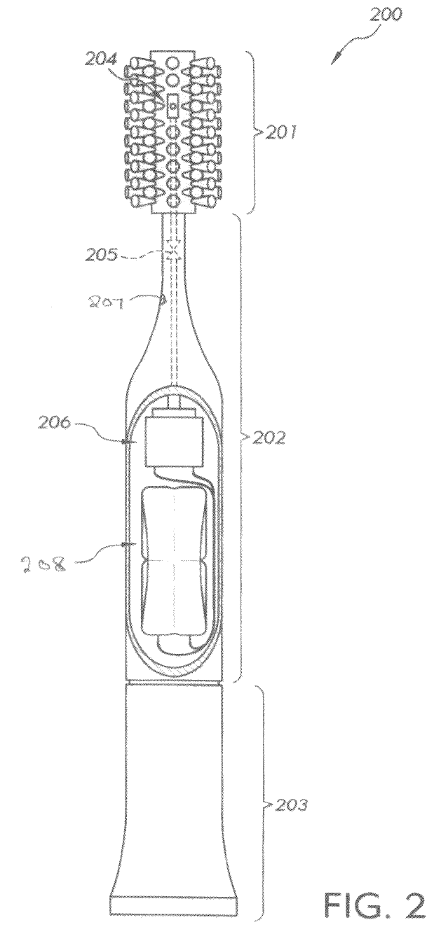

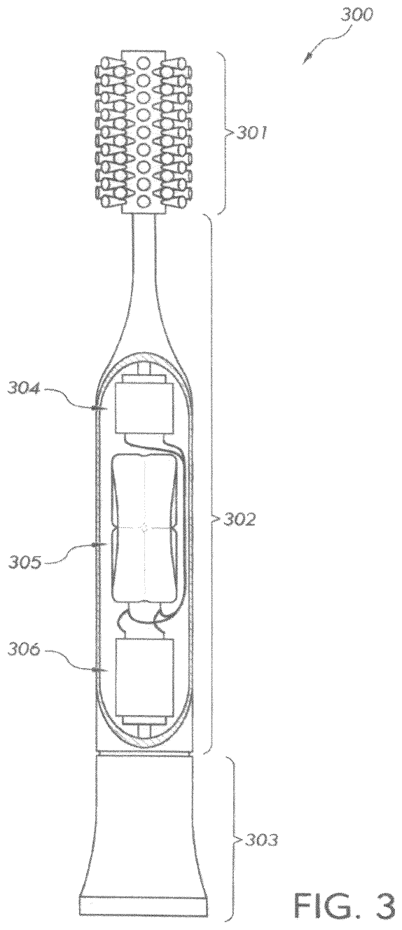

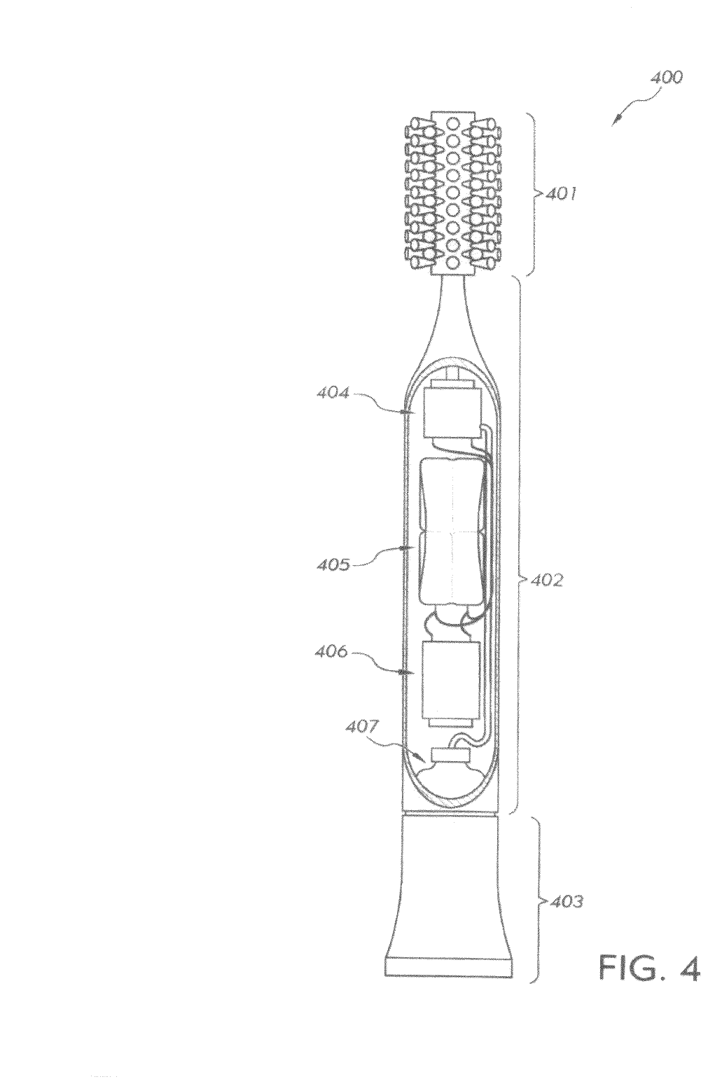

Referring now to FIGS. 1-4, an enhanced oral care implement 100, 200, 300, 400 will now be discussed in more detail. Described herein is an oral care implement that may comprise a head component 101, 201, 301, 401. The head component may be cylindrical or substantially cylindrical in shape. Alternatively, the head component may be elliptical, square, rectangular, or virtually any other shape that may be conducive to sound teeth cleaning and otherwise cleaning of a user's oral cavity, which shapes would be readily known by those skilled in the art.

The head component may be comprised of any suitable material non-toxic to humans, dogs, cats, and other animals when placed in a their oral cavity (i.e., their mouth). Such materials may include, without limitation, nylon, plastic, or aluminum or other suitable metals, which would be readily ascertained by those skilled in the art.

In one non-limiting embodiment provided by way of example only, the head component, when comprising a rectangular shape, may be between approximately 0.25 inches and 1 inch wide, and between approximately 0.5 and 5.1 inches long. In another non-limiting embodiment, when the head component is cylindrical, the diameter of the head component may be between approximately 0.25 inches and 2 inches, the circumference of the head component may be between approximately 0.79 and 6.28 inches, and the length of the head component may be between approximately 0.5 and 5.1 inches long. The foregoing dimensions are intended to serve as non-limiting illustrations. The head component may comprise any dimensions that are consistent with effective operation of oral care implements as described herein. The head component should be sized such that utilization of the enhanced oral care implement in a user's oral cavity does not create substantial discomfort for the user. Accordingly, the illustrative dimensions identified above may be reduced in a manner proportionate to the reduced area of the user's oral cavity for embodiments intended specifically for use by children.

The head component may comprise a wall element. Bristles 104 may be attached to the wall element of the head component and may extend outward from the wall element. In an embodiment, the head component may be cylindrical or substantially cylindrical in shape and the bristles may protrude outward from a cylindrical or substantially cylindrical wall element of the head component. These outward protruding bristles may be located around the entire circumference of the cylindrical or substantially cylindrical head component. According to such embodiment, the bristles may be between approximately 2.3 millimeters and 19.2 millimeters in length, without limitation.

The bristles may be made of any suitable material that is non-toxic to humans, dogs, cats, and other animals when placed in their oral cavity, which materials would be readily identifiable by those skilled in the art. Without limitation, such materials include nylon. The bristles may be soft, medium, or hard. In a preferred embodiment, the bristles may be soft to minimize the potential damage to tooth enamel and to gums potentially caused by hard bristles. The bristles may or may not be multi-layered. The bristles may or may not be angled.

Enhanced oral care implements as described herein may comprise a neck component 102, 202, 302, 402 attached to the head component at a first end and attached to a handle component 103, 203, 303, 403 at a second end.

The neck component may comprise a motor element 306, 406. The motor element may comprise a stepper motor, a hollow cup motor, a DC brush motor (e.g., a micro DC brush motor), or a DC brushless motor, without limitation.

In an embodiment, where the head component is rectangular or substantially rectangular in shape and approximates that of a conventional toothbrush with bristles extending outward from a planar or substantially planar wall, the motor element may vibrate or oscillate the head component, such as in a back-and-forth motion, without limitation. Such vibration or oscillation, which in some embodiments may be effectuated using sonic pulse technology, as would be readily understood by those skilled in the art, may enhance effective tooth brushing such as by facilitating the removal of plaque from a user's teeth. The rate at which the vibration or oscillation occurs may be adjustable by the user. The rate at which the vibration or oscillation occurs may be managed by the user through manipulation of a control aspect (e.g., a switch, button, or other similar operational component), which may be positioned anywhere on the oral care implement as long as such positioning does not affect a user's ability to handle and operate the oral care implement. Those of skill in the art will readily appreciate suitable vibration and oscillation rates conducive to effective use of an oral care implement. The vibration or oscillation may occur at a rate of between 10,000 and 50,000 strokes per minute, without limitation.

In an embodiment, where the head component is cylindrical or substantially cylindrical in shape 101, and comprises bristles protruding outward from a cylindrical or substantially cylindrical wall element, the motor element may cause the head component to rotate around a vertical axis. Such rotation may be 360 degrees. In an alternative embodiment, the motor may cause the head component to rotate around a vertical axis 180 degrees in a back-and-forth sequence. The rate at which the rotation occurs may be adjustable by the user. The rate at which the rotation occurs may be managed by the user through manipulation of a control aspect (e.g., a switch, button, or other similar operational component), which may be positioned anywhere on the oral care implement as long as such positioning does not affect a user's ability to handle and operate the oral care implement. Those of skill in the art will readily appreciate suitable rates of rotation conducive to effective use of an oral care implement. Without limitation, such rates of rotation around a vertical axis may between 2,500 and 7,500 rotations per minute.

The neck component may comprise a power source 208, 305, 405. The power source may be rechargeable. The power source may comprise a lithium-ion re-chargeable battery or a non-rechargeable lithium, alkaline, or NiMH battery. The foregoing examples of suitable power sources are not intended to be limiting. Those of skill in the art will readily recognize suitable power sources for use in enhanced oral care implements as described herein.

In an embodiment, the power source may be replaceable. According to such an embodiment, it is intended that the oral care implement continue to be used upon replacement of the power source. According to such an embodiment, the power source may be contained within a power source holder such as a battery holder. In such an embodiment, the interior of the power source holder may be accessible from the outer surface of the oral care implement.

In an alternative embodiment, the power source may not be replaceable. According to such embodiment the power source holder comprising a power source may be built into an oral care implement during manufacturing and may not be accessible from the exterior of the enhanced oral care implement. According to such an embodiment, the oral care implement may either be disposed of upon depletion of the useful battery life or may be continued to be used as a conventional toothbrush for a period consistent with dental industry guidelines concerning an appropriate amount of time to use a toothbrush.

In an embodiment, the power source may be housed together with a motor element in a housing located within the neck component, 202, 302, 402.

The neck component may further comprise a reservoir element 407 configured to hold a solution. This solution may comprise one or more of the following substances, without limitation: water, an antiseptic solution, dentifrice, fluoride, and hydrogen peroxide. In one non-limiting embodiment, the reservoir element may be designed to hold between approximately 0.25 ounces and 4 ounces of liquid, preferably between 1.5 and 2 ounces. The reservoir element may be accessible through an aperture located on the outer surface of the neck component. The aperture may be reversibly opened and closed such as with a sliding or a removable cover, without limitation. In an embodiment, the solution may be added to the reservoir element through the aperture when the aperture is in the open position.

In an alternative embodiment, the reservoir element may be accessed by detaching the neck component either at the first end or at the second end of the neck component. According to such embodiment, the solution may be added to the reservoir element by pouring the solution in at an open end of the neck component. Once solution has been added to the reservoir element, the neck component may be re-attached to the head component or the handle component, as appropriate.

The head component further comprises a nozzle element 204. The nozzle element may be located anywhere on the head component, however preferably the nozzle element is positioned so that when solution is ejected through the nozzle element the solution is transmitted in the same direction as that in which the bristles extend. Such positioning may facilitate transmission of solution into an oral cavity of a user of an enhanced oral care implement as described herein.

The nozzle element may have any sized diameter that is conducive to effective operation of the enhanced oral care implements described herein. Without limitation, the diameter of the nozzle element may be between approximately 1 millimeter and 4 millimeters. In an embodiment, the nozzle element may be adjustable in size.

The nozzle element may be operatively connected with a valve element 205. Solution propelled up from the reservoir element to the nozzle element may traverse the valve element and thereafter be propelled through the nozzle element and into the immediate surrounding environment.

Solution housed within the reservoir element may be propelled up to and through the valve element using a pump element 206, 304, 404. The pump element may comprise a tubular aspect 207 that may be partially submerged at a first end in solution housed within the reservoir element. The tubular aspect may extend upward through the neck component and into the head component where it may connect to the valve element. In this way, solution housed within the reservoir element may be propelled from the reservoir element, through the tubular aspect, through the valve element, and out into the immediate environment through the nozzle element.

In an embodiment, the pump element may be activated by applying pressure to a button or other similar control aspect on an exterior surface of an enhanced oral care implement as described herein. Upon activation, the pump element may be triggered to propel solution from the reservoir element up through the tubular aspect, through the valve element, and out through the nozzle element. Those of skill in the art will readily appreciate the foregoing manual pumping mechanism is intended to operate in substantially the same manner as a conventional spray bottle.

When solution is projected out through the nozzle element, it may mix with dentifrice, which may be located on the bristles of the enhanced oral care element. In this way, where the solution comprises a pure aqueous solution (i.e., water), a dentifrice-water solution may then be propelled out into the oral cavity of a user of the enhanced oral care implement.

In this same connection, and according to an alternative embodiment, the solution in the reservoir element may comprise an antiseptic solution. According to such embodiment, the solution projected through the nozzle element and into a user's oral cavity may constitute a dentifrice-antiseptic solution.

While much of the discussion herein contemplates use of the enhanced oral care implements described herein by human beings, those of ordinary skill in the art will readily discern that, human beings may use oral care implements as described herein to clean teeth and otherwise to clean the oral cavities of many other animals having an oral cavity comprising teeth. Such other animals include, without limitation, pets such as dogs and cats. The operability of the enhanced oral care implements described herein will be substantially identical when such enhanced oral care implements are used to clean the teeth and otherwise the oral cavities of such other animals.

The valve element may operatively connect the head component with the reservoir element. A pump element 206, 304, 404 may operatively connect the reservoir element with the nozzle element through the valve element. Solution housed within the reservoir element may be propelled through the nozzle element by the pump element, which may propel the solution through a tubular aspect, then through the valve element, and finally through the nozzle element. The solution may then be ejected out of the nozzle element into the surrounding environment, which may be a user's oral cavity.

The handle component may be comprised of plastic, rubber, or otherwise suitable material as would be readily known by those skilled in the art. In a preferred embodiment, the handle component may be comprised at least in part of rubber to facilitate gripping by a user of the enhanced oral care implements described herein.

The handle component may be any length that is conducive to operation of the enhanced oral care implements described herein. Without limitation, the handle component may be between approximately three inches and nine inches in length.

The handle component may be straight or contoured. In an embodiment, the handle component may have a curvature that facilitates gripping by a user of an enhanced oral care implement as described herein.

The handle component may be detachable from the neck component or may be contiguous with the neck component.

In an embodiment wherein the handle component is detachable from the neck component, the end of the handle component proximal the neck component may comprise a light emitting diode (LED). The LED may be used, without limitation and by way of example only, to illuminate the interior of a user's oral cavity to facilitate effective flossing and/or to aid in visual inspection of the oral cavity.

In an embodiment where the handle component is detached from the neck component, the handle component may further comprise a tongue scraper aspect. The tongue scraper aspect may, without limitation, be positioned at an end of the handle component proximal the neck component. Those of skill in the art will readily appreciate the utility of tongue scrapers, which includes, without limitation, removal of bacteria from a user's tongue thereby reducing risk of cavity formation and contributing to fresher breath. The tongue scraper aspect, however, need not be positioned on the handle component but rather may be positioned anywhere else on or in the enhanced oral care implements described herein as long as such positioning is consistent operation of the enhanced oral care implement. In one alternative non-limiting embodiment, where an enhanced oral care implement as described herein comprises a rectangular or substantially rectangular head component, the tongue scraper may be positioned on the side of the head component opposite the bristles. In the case of a cylindrical or substantially cylindrical head component, the tongue scraper aspect may be positioned at or near the top of the head component.

Consistent with the above description of the enhanced oral care implements contemplated herein, and as described above, enhanced oral care implements described herein may be utilized in connection with methods for cleaning oral cavities of human beings and other animals. Such methods include, without limitation, utilizing the functionality of the enhanced oral care implements described herein to clean effectively the teeth and otherwise the oral cavities of human beings and other animals.

While various aspects have been described in the above disclosure, the description of this disclosure is intended to illustrate and not limit the scope of the claims. The systems and methods enabled by this disclosure are defined by the scope of the claims and not the illustrations and examples provided in the above disclosure. Skilled artisans will appreciate additional aspects of the invention, which may be realized in alternative embodiments, after having the benefit of the above disclosure. Other aspects, embodiments, and modifications are within the scope of this disclosure.

What is claimed is:

1. An enhanced oral care implement comprising:
a substantially cylindrical head component comprising bristles extending outward around the circumference of the head component from a wall element;
a neck component comprising a motor element and a power source; and,
a handle component.

2. The enhanced oral care implement of claim 1, wherein the neck component further comprises a reservoir element.

3. The enhanced oral care implement of claim 2, wherein the neck component further comprises a pump element that includes a tubular aspect, and a valve element.

4. The enhanced oral care implement of claim 3, wherein the head component further comprises a nozzle element.

5. The enhanced oral care implement of claim 4, wherein the handle component further comprises a tongue scraper aspect.

6. The enhanced oral care implement of claim 5, wherein the handle component further comprises one or more light emitting diodes.

7. A method for cleaning the oral cavity of a human being comprising:
providing the enhanced oral care implement of claim 1; and
using the enhanced oral care implement to clean the human being's oral cavity.

8. The method of claim 7, wherein the neck component of the enhanced oral care implement further comprises a reservoir element.

9. The method of claim 8, wherein the neck component further comprises a pump element that includes a tubular aspect and a valve element.

10. The method of claim 9, wherein the head component of the enhanced oral care implement further comprises a nozzle element.

11. The method of claim 10, wherein the handle component of the enhanced oral care implement further comprises a tongue scraper aspect.

12. The method of claim 10, wherein the handle component of the enhanced oral care implement further comprises one or more light emitting diodes.

13. The method of claim 11, further comprising the step of scraping an individual's tongue using the tongue scraper.

14. The method of claim 12, further comprising the step of using the one or more light emitting diodes to visually inspect an individual's oral cavity.
